(19) United States
(12) Patent Application Publication
(10) Pub. No.: US 2022/0196631 A1
(43) Pub. Date: Jun. 23, 2022

(54) DEVICES AND METHODS FOR DETECTING ANALYTES USING FUNCTIONALIZED CARBON ALLOTROPES

(71) Applicant: GRAPHENE-DX, INC., Boston, MA (US)

(72) Inventors: Namal NAWANA, Weston, MA (US); Mehdi ABEDI, Brighton, MA (US); Mohammad E. TASLIM, Needham, MA (US); Mohammed FOTOUHI, Weston, MA (US); Edward A. GREENFIELD, Stoughton, MA (US)

(21) Appl. No.: 17/542,402

(22) Filed: Dec. 4, 2021

Related U.S. Application Data
(60) Provisional application No. 63/121,718, filed on Dec. 4, 2020.

(51) Int. Cl. G01N 33/487

(52) U.S. Cl. CPC G01N 33/48714; B82Y 5/00

(57) ABSTRACT

The present teachings are generally directed to sensors that employ antibody-functionalized graphene nano-flakes (and/or a graphdiyne layer) for detecting a variety of analytes in a variety of samples. A plurality of graphene nano-flakes (and/or a graphdiyne layer) can be deposited on a underlying substrate, e.g., in the form of a single layer or multiple stacked layers, and functionalized with an antibody that specifically binds with an analyte of interest. A sample under investigation can be introduced onto the antibody-functionalized graphene nano-flakes (and/or a graphdiyne layer). The interaction of the analyte of interest, if present in the sample, with the antibody-functionalized graphene nano-flakes (and/or a graphdiyne layer) can mediate a change in at least one electrical property of the graphene nano-flakes, e.g., their DC electrical resistance. An analyzer can detect such a change and analyze it to determine whether the analyte is present in the sample. In some embodiments, calibration methods can be employed to quantify the analyte present in the sample.

### RELATED APPLICATION

This application claims priority to U.S. Provisional Patent Application No. 63/121,718, filed Dec. 4, 2020, entitled “Devices and Methods For Detecting Analytes Using Functionalized Carbon Allotropes,” which is hereby incorporated by reference in its entirety.

### TECHNICAL FIELD

The present invention relates generally to systems and methods for detecting, and optionally quantifying, analytes such as pathogens, allergens, and biomarkers based on antibody-and/or aptamer-functionalized allotropes of carbon, such as graphene nano-flakes and graphdiyne.

### BACKGROUND

Conventional methods for detecting and quantifying analytes, such as pathogens, biomarkers in bodily fluids, include immunoassays, such as ELISA, that utilize a variety of detection techniques, such as fluorometric detection, chemiluminescence detection, electrochemical detection, etc. The conventional methods can be time-consuming and/or can require complex sample preparation.

Accordingly, there is a need for improved systems and methods for detecting analytes, such as pathogens, allergens and biomarkers in a variety of samples, such as food samples and biological samples, such as bodily fluid samples.

### SUMMARY

In one aspect, a sensor for detecting an analyte in a sample is disclosed, which comprises a plurality of graphene nano-flakes deposited on an underlying substrate, a plurality of antibodies and/or aptamers coupled to the graphene nano-flakes to generate a plurality of antibody- and/or aptamer-functionalized graphene nano-flakes, wherein the antibodies and/ or aptamers exhibit specific binding to the analyte, and a plurality of electrical conductors electrically coupled to the graphene nano-flakes for measuring an electrical property thereof.

A sensor according to the present teachings can be employed to detect a variety of different analytes in a sample. For example, a sensor according to the present teachings can be used to detect pathogens, allergens, and/or biomarkers in a sample, including a food sample and a biological sample, among others. For example, a sensor according to the present teachings can be used to detect listeria monocytogene, E. coli, chlamydia, and gonorrhea bacteria in a sample, e.g., a biological sample. In other embodiments, a sensor according to the present teachings can be employed to detect allergens, such as gluten proteins, in a sample. In yet other embodiments, a sensor according to the present teachings can be employed to detect a biomarker, such as troponin, C reactive protein (CRP), and B-type natriuretic peptide (BNP), in a sample (e.g., a biological sample). In yet other embodiments, a sensor according to the present teachings can be used to detect viruses, such as the influenza virus, Corona viruses (e.g., SARS-CoV-2 virus), among others.

In some embodiments, the sensor can include a reference electrode that is disposed n proximity of the antibody- and/or aptamer-functionalized graphene nano-flakes. An AC voltage source can be used to apply an AC voltage to the reference electrode. In some embodiments, the frequency of the applied AC voltage can be in a range of about 1 kHz to about 1 MHz, and the amplitude of the applied AC voltage can be in a range of about 100 millivolts to about 3 volts. In some embodiments, a DC ramp voltage is applied to the AC electrode, together with the AC voltage, during data acquisition. The DC ramp voltage can be, for example, from about −10 volts to about 10 volts.

In some embodiment, the measured electrical property of the functionalized graphene nano-flakes can be the DC electrical resistance of the graphene nano-flakes. By way of example, a detected change in the mobility of electrons in the functionalized nano-flakes in response to specific binding of a target analyte to the antibodies/aptamers can be employed to detect the target analyte.

The graphene nano-flakes can be deposited on a variety of different substrates. By way of example, in some embodiments, the substrate can be a semiconductor substrate, such as silicon. In other embodiments, the substrate can be a glass substrate. In yet other embodiments, the substrate can be a polymeric substrate (e.g., a plastic substrate).

In a related aspect, a method of detecting an analyte in a sample is disclosed, which comprises applying the sample to a plurality of graphene nano-flakes functionalized with an antibody/aptamer exhibiting specific binding to the analyte, measuring at least one electrical property of the functionalized graphene nano-flakes, and using the measured electrical property to determine whether the analyte is present in the sample.

In some embodiments, the method can further include quantifying the analyte, if present in the sample.

In another aspect, a sensor for detecting an analyte in a sample is disclosed, which comprises a graphdiyne layer deposited on an underlying substrate, a plurality of antibodies and/or aptamers coupled to the graphdiyne layer to generate a functionalized graphdiyne layer, wherein the antibodies and/or aptamers exhibit specific binding to the analyte, and a plurality of electrical conductors electrically coupled to the graphdiyne layer for measuring an electrical property thereof. In some embodiments, the graphdiyne layer can comprise a plurality of graphdiyne flakes.

The systems and methods according to the present teachings can be employed to investigate a variety of different samples, such as biological samples, food samples, among others.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes, and may not have been selected to delineate or circumscribe the inventive subject matter.

### BRIEF DESCRIPTION OF THE DRAWINGS

So that the present disclosure can be understood in greater detail, a more particular description may be had by reference to the features of various embodiments, some of which are illustrated in the appended drawings. The appended drawings, however, merely illustrate pertinent features of the present disclosure and are therefore not to be considered limiting, for the description may admit to other effective features.

FIG. 1 schematically depicts a sensor according to an embodiment, which includes a plurality of graphene nano-flakes deposited over a substrate, in accordance with some embodiments.

FIG. 2 schematically depicts an analyzer suitable for measuring electrical resistance of the sensor depicted in FIG. 1, in accordance with some embodiments.

FIG. 3 schematically depicts a device for measuring the electrical resistance of the sensor depicted in FIG. 1, in accordance with some embodiments.

FIG. 4A schematically depicts a sensor according to an embodiment, which includes a reference electrode positioned in proximity of the graphene nano-flakes, in accordance with some embodiments.

FIG. 4B schematically depicts a combination of a ramp voltage and an AC voltage applied to the reference electrode of the sensor, in accordance with some embodiments.

FIGS. 5A and 5B schematically depict a sensor according to an embodiment suitable for detecting a pathogen in a sample, in accordance with some embodiments.

FIG. 6 schematically depicts an embodiment of the sensor depicted in FIGS. 5A and 5B in which a reference electrode is positioned in proximity of antibody-functionalized graphene nano-flakes, in accordance with some embodiments.

FIG. 7 schematically depicts a sensor according to an embodiment, which can be used to detect biomarkers in a sample, in accordance with some embodiments.

FIG. 8 schematically depicts a graphene layer according to an embodiment, which comprises a plurality of graphene and/or graphdiyne flakes deposited on a graphene or graphdiyne layer, in accordance with some embodiments.

FIG. 9 schematically depicts a plurality of electrically conductive pads and associated electrical paths utilized in the sensor of FIG. 6 for measuring electrical resistance of the graphene nano-flakes, in accordance with some embodiments.

FIG. 10 schematically depicts an embodiment of a sensor according to the present teachings, which includes an analysis sensing unit and a calibration sensing unit, in accordance with some embodiments.

FIG. 11 schematically depicts an embodiment of a sensor according to the present teachings, which includes a plurality of sensing units, in accordance with some embodiments.

FIGS. 12A and 12B depict an embodiment of a sensor according to the present teachings, which includes a microfluidic device for delivering a sample onto a sensing unit comprises antibody-functionalized graphene nano-flakes, in accordance with some embodiments.

FIG. 13 schematically depicts a sensor according to an embodiment, which includes a reference electrode to which an AC voltage can be applied, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DETAILED DESCRIPTION

The present teachings are generally directed to sensors that employ functionalized allotropes of carbon (e.g., graphene flakes and/or graphdiyne) for detecting a variety of analytes in a variety of samples. As discussed in more detail below, in many embodiments, a plurality of graphene nano-flakes or graphdiyne can be deposited on a underlying substrate, e.g., in the form of a single layer or multiple stacked layers, and functionalized with an antibody and/or an aptamer that specifically binds to an analyte of interest. A sample under investigation can be introduced onto the functionalized graphene flakes or graphdiyne. The interaction of the analyte of interest, if present in the sample, with the functionalized graphene flakes or graphdiyne can mediate a change in at least one electrical property of the graphene flakes or graphdiyne, e.g., the electron mobility that can manifest itself as a change in their DC electrical resistance. An analyzer can detect such a change and analyze it to determine whether the analyte is present in the sample. In some embodiments, calibration methods can be employed to quantify the analyte present in the sample.

The terms “graphene nano-flake,” “graphene flake,” and “graphene nanodot” are used herein interchangeably and refer to a plurality of hexagonal sp2-hybridized carbon rings that are fused together. In some embodiments, a plurality of graphene nano-flakes can be distributed so as to form a single layer while in other embodiments, a plurality of graphene nano-flakes are distributed such that at least some of the graphene nano-flakes are stacked on one another.

The term “analyte” as used herein refers to any molecular species whose detection in a sample is desired. For example, an analyte can be a protein, such as an antigen, or a pathogen, such as a bacterium.

The term “antibody” as used herein refers to a polypeptide exhibiting specific binding affinity, e.g., an immunoglobulin chain or fragment thereof, comprising at least one functional immunoglobulin variable domain sequence. An antibody encompasses full length antibodies and antibody fragments. In some embodiments, an antibody comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody.

The term “antibody” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

In some embodiments, an antibody is a glycoprotein produced by B lymphocytes in response to stimulation with an immunogen. An antibody can be composed of 4 polypeptides—2 heavy chains and 2 light chains—bound together by disulfide bonds to form a Y-shaped molecule.

The term “aptamer,” as used herein, refers to an oligonucleotide or a peptide molecule that exhibits specific binding to a target molecule. Aptamers are typically created by selecting them from a large random pool of oligonucleotide or peptide sequences, but natural aptamer do also exist.

The term “about” as used herein to qualify a numerical value is intended to denote a maximum variation of 10% about the numerical value.

Graphdiyne is a 2D carbon allotrope composed of sp and sp² hybridized carbon atoms, which can be constructed by replacing some carbon-carbon bonds in graphene with uniformly distributed diacetylenic linkages.

Without any loss of generality, in the following embodiments, antibodies are employed for functionalizing a graphene flake or a graphdiyne layer. In all of these embodiments, rather than antibodies, aptamers can be employed for functionalizing the graphene flake or the graphdiyne layer.

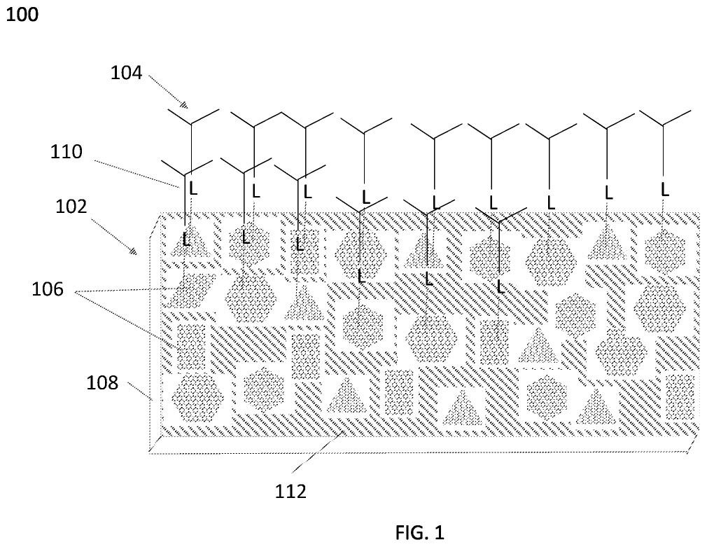

FIG. 1 schematically depicts an embodiment of a sensor 100 according to the present teachings that can be used for detecting an analyte in a sample, which includes a sensing element 102 having a plurality of graphene flakes 106 that are deposited on an underlying substrate 108, where the graphene flakes are functionalized with a plurality of antibodies 104. The graphene flakes can have a variety of different shapes, such as those depicted in FIG. 1. However, it should be understood that the shapes of the graphene flakes are not limited to the polygonal shapes depicted herein, and that the graphene flakes can have a variety of shapes.

In some embodiments, the graphene flakes can be distributed over a surface area in a range of about 1 mm² to about 1 cm². By way of example, the graphene flakes can be distributed over a rectangular surface area having a length in a range of about 1 mm to about 1 cm and a width in a range of about 1 mm to about 1 cm, though other dimensions can also be utilized.

Alternatively or additionally, the sensor 100 according to the present teachings can include a graphdiyne-based sensing element. In some embodiments, such a graphdiyne-based sensing element can include a graphdiyne layer and/or a plurality of graphdiyne flakes, which are deposited on an underlying substrate 108. The graphdiyne-based sensing element can be functionalized with a plurality of antibodies. In all embodiments disclosed in the present teachings, the graphene flakes can be partly or entirely substituted with graphdiyne flakes.

With continued reference to FIG. 1, in this embodiment, the graphene flakes layer and/or a graphdiyne layer 106 is functionalized with the antibodies 104 that exhibit specific binding to an analyte of interest. In this embodiment, a linker 110 is employed to couple the antibodies 104 to the graphene flakes and/or graphdiyne layer 106. The linker can be coupled at one end thereof to the graphene flakes and/or graphdiyne layer 106 via π-π interaction and can be attached to the antibody via a covalent bond at another end thereof.

A variety of linkers can be employed in the practice of the invention. By way of example, in some embodiments, the linker can be 1-pyrenebutonic acid succinimidyl ester. It has been discovered that this linker can be used to attach a variety of different antibodies to a graphene film, and hence it is expected that it can be similarly employed to attach a variety of different antibodies (or aptamers) to graphene flakes and/or a graphdiyne layer.

In some embodiments, the sensor 100 includes a passivation layer 112 that is disposed on the substrate to cover the areas of the substrate surface that are free of graphene flakes (and/or graphdiyne) and/or cover graphene flakes (and/or portions of the graphdiyne layer) that are not functionalized with antibodies. In some embodiments, the passivation layer can be formed using Tween 20, BLOTTO, BSA (Bovine Serum Albumin), and/or gelatin, amino-PEGS-alcohol (pH 7.4). Further details regarding suitable linkers and passivating agents can be found, e.g., in U.S. Pat. No. 9,664,674 B2, which is herein incorporated by reference in its entirety.

In some embodiments, the electrode of the sensor can include a graphene layer, a plurality of graphene flakes disposed with a passivation layer, a graphdiyne layer, a plurality of graphdiyne flakes disposed with a passivation layer, or any combination thereof, disposed on the substrate. Furthermore, the term “graphene layer” as used herein can refer to a planar structure of graphene, a deposited layer that comprises a plurality of graphene flakes, a planar structure of graphdiyne, a deposited layer that comprises a plurality of graphdiyne flakes, or any combination thereof. For example, the electrode of the sensor can include a multi-layer structure including a plurality of graphene and/or graphdiyne flakes deposited on a graphene or graphdiyne layer. The term “graphene layer” can also be collectively used as including a passivation layer.

As noted above, the graphene flakes 106 (and/or a graphdiyne layer) can be functionalized with a plurality of antibodies 104 that exhibit specific binding to an analyte of interest. By way of example, the graphene flakes 106 (and/or a graphdiyne layer) can be functionalized with an antibody 104 that exhibits specific binding to a gluten protein, such as gliadin. By way of example, in some embodiments, the anti-gliadin antibody can be 14D5 antibody marketed by Abcam for selectively binding to the gliadin protein.

Further, the graphene flakes 106 (and/or a graphdiyne layer) can be functionalized with a plurality of antibodies 104 that exhibit specific binding to a biomarker of interest. By way of example, the graphene flakes 106 (and/or a graphdiyne layer) can be functionalized with an antibody 104 that exhibits specific binding to any of troponin, e.g., a particular isoform of troponin, C-reactive protein, B-type natriuretic peptide, or myeloperoxidase. In some embodiments, the anti-biomarker antibodies are monoclonal antibodies that exhibit specific binding to a particular isoform of the biomarker, e.g., a specific isoform of troponin. In other embodiments, the anti-biomarker antibodies can be polyclonal antibodies that exhibit binding to multiple isoforms of the biomarker. By way of example, in some embodiments, the graphene flakes 106 ((and/or a graphdiyne layer) can be functionalized with cardiac troponin T (cTnT) and/or cardiac troponin I (cTnI).

In some embodiments, the graphene flakes 106 (and/or a graphdiyne layer) is functionalized with an antibody 104 that can specifically bind to a pathogen of interest. By way of example, in some embodiments, the graphene flakes 106 can be functionalized with listeria monocytogenes antibody LZF7 (BGN/0884/67), which is an IgG2a mouse anti-listeria monocytogenes monoclonal antibody. In another embodiment, the graphene flakes 106 (and/or a graphdiyne layer) can be functionalized with an anti-chlamydia antibody, such as monoclonal or polyclonal antibodies. A number of anti-chlamydia antibodies are commercially available. For example, anti-chlamydia antibody marketed by Genetex under Cat #GTX40387 can be employed. In another embodiment, the graphene flakes 106 (and/or a graphdiyne layer) can be functionalized with anti-gonorrhea antibodies, e.g., monoclonal or polyclonal antibodies. For example, anti-gonorrhea antibody marketed by Creative Diagnostics under Cat #DMZB9759 can be employed. Yet in another embodiment, the graphene flakes 106 (and/or a graphdiyne layer) can be functionalized with anti-HPV (human papilloma virus) antibodies. For example, anti-HPV antibody marketed by Abcam under Cat #ab2417 or by Thermo Fisher under Cat #MA512446 can be employed.

In some embodiments, the graphene flakes 106 (and/or a graphdiyne layer) is functionalized with an antibody 104 that exhibits specific binding to hemoglobin protein, e.g., human hemoglobin protein. By way of example, an anti-human hemoglobin antibody can be obtained from Sigma Aldrich under the product code H4890-2ML.

In some embodiments, the graphene flakes 106 (and/or a graphdiyne layer) can be functionalized with protein G (PrG), which can be coupled to the underlying graphene layer via pi-pi interaction. The antibodies 104 can be then covalently attached to the PrG. In some embodiments, the PrG can advantageously orient the antibody molecules so as to enhance the detection of a target analyte (such as a virus (e.g., the SARS-CoV-2 virus)).

In some embodiments, rather than functionalizing the graphene flakes 106 (and/or a graphdiyne layer) with antibodies 104 that exhibit specific binding to at least one protein associated with the virus of interest, the graphene flakes 106 can be functionalized with one or more proteins associated with the virus so as to detect antibodies generated by an infected person in a biological sample extracted from such a person. A person's immune system may release antibodies (IgM) subclass upon infection. As the infection develops, this response changes mainly to an IgG subclass response. These antibodies can bind to the viral proteins. By way of example, in some such embodiments, the graphene flakes 106 can be functionalized with N and/or S protein associated with SARS-CoV-2virus. For example, SARS-CoV-2 antibodies can be obtained from Sino Biological under Cat #40143-MM05, from GenScript under Cat #6D11F2 and 3F9C12, from Atlas Antibodies under Cat #AMAb91262, from GeneTex under Cat #GTX135357. Further, a saliva sample may contain IgA antibodies.

In some embodiments, the graphene flakes (and/or a graphdiyne layer) deposited on an underlying substrate (e.g., plastic, or semiconductor, such as silicon) can be incubated with the linker molecule (e.g., a 5 mM solution of 1 pyrenebutonic acid succinimidyl ester) for a few hours (e.g., 2 hours) at room temperature. The linker modified graphene flakes (and/or a graphdiyne layer) can then be incubated with an antibody (e.g., 14D5) in a buffer solution (e.g., NaCO₃-NaHCO₃ (pH 9) at a selected temperature and for a selected duration (e.g., 7-10 hours at 4° C.), followed by rinsing with deionized (DI) water and phosphate buffered solution (PBS). In some cases, in order to quench the unreacted succinimidyl ester groups, the antibody-functionalized graphene flakes can be incubated with ethanolamine (e.g., 0.1 M solution at a pH of 9 for 1 hour).

Without being bound to any particular theory, the interaction between a gluten protein and the antibody-functionalized graphene flakes 106 can result in a change in at least one electrical property of the underlying layer of graphene flakes or graphdiyne, such as the electrical resistance of the underlying layer of graphene flakes.

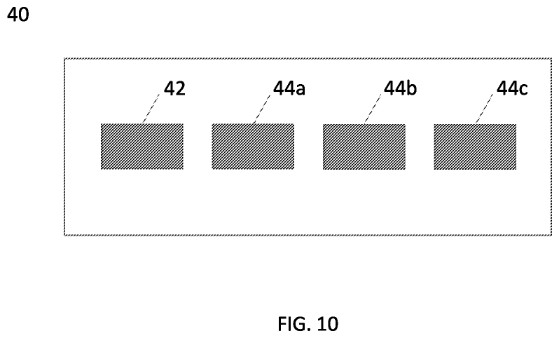

A plurality of electrode pads, such as those depicted in FIG. 9 can be coupled, via electrically conductive paths, to the antibody-functionalized graphene flakes (and/or a graphdiyne layer) to allow the measurement of an electrical resistance thereof.

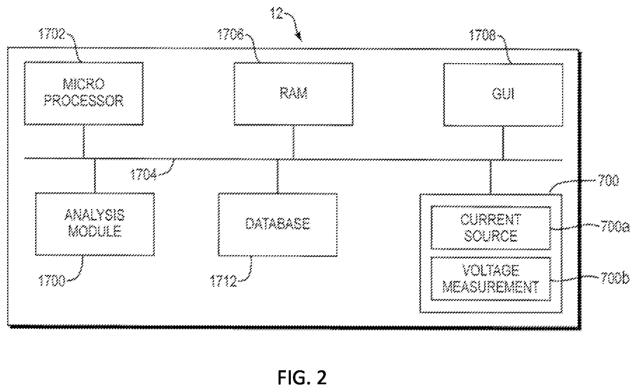

As shown in FIG. 2, an analyzer 12 can measure such a change in the electrical resistance of the layer of graphene flakes (and/or a graphdiyne layer) and determine whether the measured change correlates with the presence of a target analyte above a certain concentration threshold in the sample under investigation. In this embodiment, the analyzer 12 includes a data acquisition unit (herein also referred to as a measurement unit) 700, and an analysis module 1700. The analyzer can also include other components, such as a microprocessor 1702, a bus 1704, a Random Access Memory (RAM) 1706, a Graphical User Interface (GUI) 1708 and a database storage device 1712. The bus 1704 can allow communication among the different components of the analyzer. In some embodiments, the analysis module can be implemented in the form of a plurality of instructions stored in the RAM 1706. In other embodiments, it can be implemented as a dedicated hardware for performing processing of data obtained by the data acquisition unit 700.

Data acquisition unit 700 may be configured to acquire electrical data from which one or more electrical properties of the sensor (e.g., its DC resistance) can be determined, in this embodiment, the data acquisition unit 700 includes a current source 700a for supplying electrical currents of selected values to the sensing elements (e.g., to the graphene nano-flakes of the sensing elements) and a voltage measuring circuit 700b that can measure the voltage across each of the sensing elements, e.g., across the graphene nano-flakes (and/or a graphdiyne layer) of each sensing element.

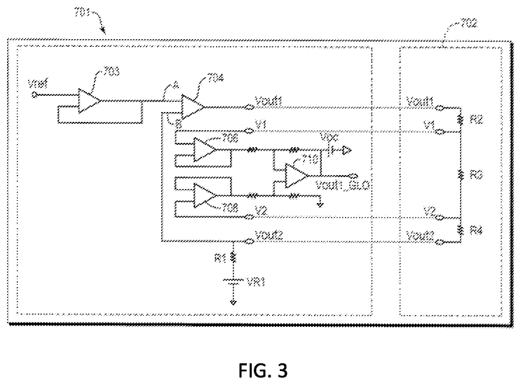

FIG. 3 schematically depicts a voltage measurement circuitry 701 according to some embodiments. Voltage measurement circuitry 701 can be employed as the measurement unit 700 for pleasuring electrical resistance of a sensor, e.g., sensor 702 that is depicted in this figure as an equivalent circuit diagram of a sensor according to the present teachings. A fixed voltage V (e.g., 1.2 V) is generated at the output of a buffer operational amplifier 703. This voltage is applied to one input (A) of a downstream operational amplifier 704 whose other input B is coupled to VR1 ground via a resister R1, The output of the operational amplifier 704 (Vout1) is coupled to one end of the sensor 702 and the non-connected to VR1 end of the resistor R1 is coupled to the other end of the sensor 702 (in this schematic diagram, resistor R2 denotes the resistance between two electrode pads at one end of a sensor, resister R3 denotes the resistance of the sensor extending between two inner electrode pads of the sensor, and resistor R4 denotes the resistance between two electrode pads at the other end of the sensor). As the operational amplifier maintains the voltage at the non-connected to VR1 end of the resistor RI, at the fixed voltage applied to its input (A), e.g., 1.2 V, a constant current source is generated that provides a constant current flow through the sensor 702 and returns to ground via the resistor R1 and VR1.

In this embodiment, a voltage generated across the sensor can be measured via the two inner electrodes of the sensor. Specifically, one pair of the inner electrode pads is coupled to a buffer operational amplifier 706 and the other pair is coupled to the other buffer operational amplifier 708. The outputs of the buffer operational amplifiers are applied to the input ports of a differential amplifier 710 whose output port provides the voltage difference across the sensor. This voltage difference (Vout1_GLO) can then be used to measure the resistance exhibited by the sensor. The current forced through R3 is set by I=(Vref−VR1)/R1. The value of VR1, is digitally, controlled. For each value of current I, the corresponding voltage (Vout1_GLO) is measured and stored. The resistance of the sensor may be different at any given current so it is calculated as derivative of voltage, Vout1_GLO, with respect to current I, i.e., R=dV/dI≈ΔV/ΔI using the stored voltage versus current I. If the sensor has linear constant resistance, the value of R can be found as R=dV/dI=ΔV/ΔI=V/I.

Referring back to FIG, 2, the analysis module 1700 can be configured to receive the current and voltage values generated and obtained by the measurement unit 700 and can process these values according to the present teachings. The analysis may identify and quantify selected species, e.g., molecular species, present in a sample. Different units in the analyzer 12, as well as other units of the analysis module, can operate under the control of the microprocessor 1702.

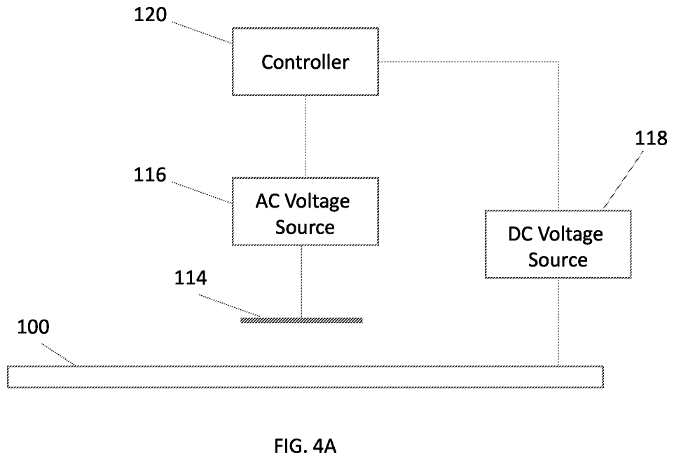

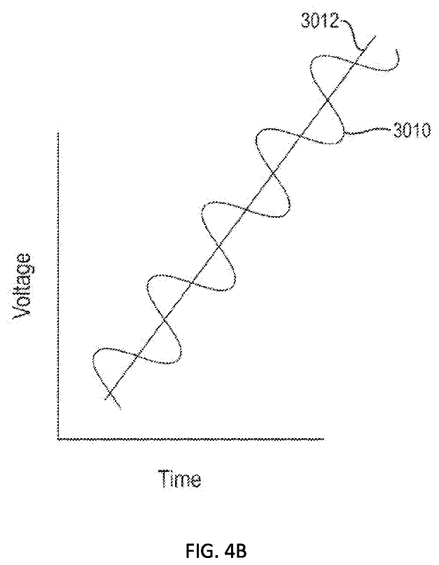

Referring to FIG. 4A, in some embodiments, the sensor 100 can include a reference electrode 114 disposed in proximity of the antibody-functionalized graphene nano-flakes (not shown in this figure for the sake of clarity), e.g., at a distance in a range of about 50 micrometers to about a few millimeters (e.g., 1-2 millimeters) above the functionalized graphene nano-flakes. In some embodiments, the distance of the reference electrode 114 relative to the antibody-functionalized graphene flakes can be in a range of about 100 microns to about 1 millimeter, or in a range of about 200 microns to about 0.5 millimeter. Further, in some embodiments, rather than being positioned above the graphene nano-flakes, the reference electrode 114 can be positioned in the same plane as the graphene layer.

The reference electrode 114 can be utilized to generate a time-varying electric field at the interface of the antibody-functionalized graphene nano-flakes and a liquid sample, e.g., a liquid sample suspected of containing an analyte, that is brought into contact with that layer. For example, in this embodiment, an AC voltage source 116 can be employed to apply an AC voltage to the reference electrode 114, which can in turn result in the generation of a time-varying electric field in the space between the reference electrode 114 and the functionalized graphene layer.

The AC reference electrode 114 can be formed of any suitable electrical conductor. Some examples of suitable conductors include, without limitation, silver, copper, and gold. In some embodiments, the thickness of the reference electrode 114 can be, for example, in a range of about 100 nm to about 400 micrometers (microns), e.g., in a range of about 1 micron to about 100 microns, though other thicknesses can also be employed.

The application of such a time-varying electric field via the reference electrode 114 to the interface between the graphene flakes and a liquid sample in contact with the graphene flakes can advantageously facilitate the detection of one or more electrical properties of the antibody-functionalized graphene flakes, e.g., a change in its resistance in response to its interaction with an analyte of interest present in the sample that exhibits specific binding to the antibody of the functionalized graphene flakes. In particular, it has been discovered that the application of an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 kHz to about 200 kHz, can be especially advantageous in this regard. By way of example, the amplitude of the AC voltage applied to the reference electrode 114 can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt.

Further, in some cases, the voltage applied to the reference electrode 114 can have an AC component and a DC offset, where the DC offset can be in a range of about −40 volts to about +40 volts, e.g., −1 volt to about +1 volt. More specifically, a controller 120 is programed to control an AC voltage source 116 and a DC voltage source 118. Although in this embodiment the AC voltage source 116 and the DC voltage source 118 are shown as two independent units, in other embodiments the functionalities of the AC voltage source 116 for applying an AC voltage and a DC offset voltage to the reference electrode 114 and the functionalities of a power supply can be combined in a single unit.

The controller 120 can be implemented in hardware, software, and/or firmware in a manner known in the art as informed by the present teachings. For example, the controller 120 can have the components illustrated in FIG. 3 for the analyzer.

By way of illustration, FIG. 4B schematically depicts a combination of an AC voltage 3010 and a DC offset voltage 3012 applied to the reference electrode 3001. By way of example, the DC offset voltage can extend from about −10 V to about 10 V (e.g., from −1 V to about 1 V), and the applied AC voltage can have the frequencies and amplitudes disclosed above.

Without being limited to any particular theory, in some embodiments, it is expected that the application of such a voltage to the reference electrode 114 can minimize, and preferably eliminate, an effective capacitance associated with a sample, e.g., a liquid sample, with which the functionalized graphene flakes are brought into contact as the sample is being tested, thereby facilitating the detection of a change in the resistance of the underlying graphene flakes in response to the interaction of the antibodies with a respective analyte. In some cases, the effective capacitance of the sample can be due to ions present in the sample.

The present teachings are not limited to detection of a gluten protein in a sample in particular, in some embodiments, the present teachings can be employed to detect one or more pathogens, e.g., bacteria, or a biomarker in a sample.

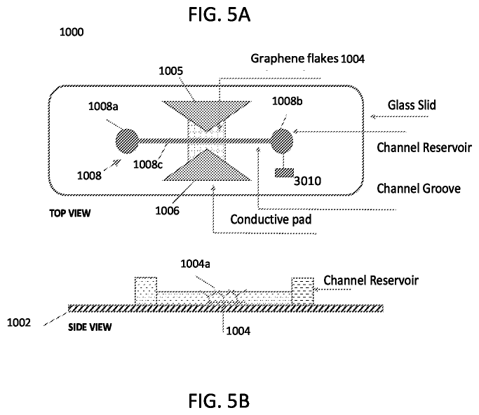

By way of example, FIGS. 5A and 5B schematically depict an example of a device 1000 according to an embodiment of the present teachings for detecting a pathogenic agent in a sample. The device 1000 includes a substrate 1002 on a top surface of which a plurality of graphene flakes 1004 (and/or a graphdiyne layer) are deposited.

A variety of different substrates can be employed. By way of example, the substrate 1002 can be any of a semiconductor, such as silicon, or glass or plastic. In some embodiments in which the substrate 1002 is formed of silicon, a layer of silicon oxide can separate the upper layer of graphene flakes from the underlying silicon layer.

The graphene flakes (and/or a graphdiyne layer) can be functionalized with an antibody 1004a that can specifically bind to a pathogen of interest. By way of example, in some embodiments, the graphene flakes can be functionalized with listeria monocytogenes antibody LZF7 (BGN/0884/67), which is an IgG2a mouse anti-listeria monocytogenes monoclonal antibody. In another embodiment, the graphene flakes (and/or a graphdiyne layer) can be functionalized with an anti-chlamydia antibody, such as monoclonal or polyclonal antibodies. A number of anti-chlamydia antibodies are commercially available. For example, anti-chlamydia antibody marketed by Genetex under Cat#GTX40387 can be employed. In another embodiment, the graphene flakes (and/or a graphdiyne layer) can be functionalized with anti-gonorrhea antibodies, e.g., monoclonal or polyclonal antibodies. For example, anti-gonorrhea antibody marketed by Creative Diagnostics under Cat#DMZB9759 can be employed. Yet, in another embodiment, the layer of graphene flakes can be functionalized with anti-HPV (human papilloma virus) antibodies. For example, anti-HPV antibody marketed by Abcam under Cat#ab2417 or by Thermo Fisher under Cat#MA5 12446 can be employed.

Similar to the previous embodiment, in some embodiments, the antibodies 1004a are coupled to the underlying graphene layer via a linker, where the linker is attached at one end thereof via π-π interactions to the graphene flakes 1004. The antibodies 1004a can be attached to the other end of the linker, e.g., via a covalent bond. By way of example, in some embodiments, 1-pyrenebutonic acid succinimidyl ester can be employed as the linker to facilitate the coupling of the antibody molecules to the underlying graphene layer. It has been discovered that 1-pyrenebutonic acid succininmidyl ester can be used to couple a variety of different antibodies to an underlying graphene layer. As such, it is expected that this linker can be used for coupling antibodies that specifically bind to a variety of different pathogens to an underlying layer of graphene flakes, where the pathogen-antibody interaction can mediate a change in one or more electrical properties of the underlying layer of graphene flakes.

In some embodiments, the graphene flakes (and/or a graphdiyne layer) can be incubated with the linker molecule (e.g., a 5 mM solution of 1-pyrenebutonic acid succimidyl ester) for a few hours (e.g., 2 hours) at room temperature to ensure covalently coupling of the linker molecules to the underlying graphene flakes. The linker modified graphene flakes can then be incubated with an antibody of interest in a buffer solution (e.g., NaCO3-NaHCO3 buffer solution (pH 9)) at a selected temperature and for a selected duration (e.g., 7-10 hours at 4° C.), followed by rinsing with deionized (DI) water and phosphate buffered solution (PBS). In order to quench the unreacted succinimidyl ester groups, the modified graphene flakes can be incubated with ethanolamine (e.g., 0.1 M solution at a pH of 9 for 1 hour).

Subsequently, the non-functionalized areas of the graphene flakes (and/or a graphdiyne layer) and/or the substrate can be passivated via a passivation layer. By way of example, the passivation of the non-functionalized portions of the graphene flakes (and/or a graphdiyne layer) and/or the substrate can be achieved, e.g., via incubation with 0.1% Tween 20 or BLOTTO, BSA (Bovine Serum Albumin), and gelatin and/or amino-PEGS-alcohol (pH 7.4). Further details regarding the use of linkers suitable for use in the practice of the invention can be found, e.g., in U.S. Pat. No. 9,664,674, which is herein incorporated by reference in its entirety.

Referring again to FIGS. 5A and 5B, two metallic conductive pads 1005/1006 in electrical contact with the layer of graphene flakes 1004 allow measuring the electrical resistance of the graphene flakes 1004, and particularly, a change in the electrical resistance of the graphene flakes 1004 in response to exposure thereof to a sample containing a pathogen, e.g., listeria, chlamydia, gonorrhea bacteria and/or HPV. In some embodiments, the electrically conductive pads 1005/1006 can be formed of silver high conductive paste, though other electrically conductive materials can also be employed. The conductive pads 1005/1006 can be electrically connected to a measurement device, e.g., a voltmeter, via a plurality of conductive wires for measuring the Ohmic electrical resistance of the graphene layer.

The device 1000 further includes a microfluidic structure 1008 having two reservoirs 1008a/1008b and a fluid channel 1008c that fluidly connects the two reservoirs 1008a/1008b. As shown more clearly in FIG. 5B, the fluid channel 1008c can be arranged such that a portion thereof is in fluid contact with a portion of the graphene flakes 1004.

In some embodiments, in use, a sample suspected of containing a target analyte (e.g., pathogen) of interest, e.g., listeria bacteria, can be introduced into one of the reservoirs 1008a/1008b and can be made to flow, e.g., via application of hydrodynamic pressure thereto, to the other reservoir through the microfluidic channel 1008c. In this embodiment, a pump 3010 can be coupled to a reservoir 1008b to facilitate the flow of the sample to the other reservoir 1008a. In other embodiments, the pump 3010 may be coupled to the other reservoir 1008a and/or to a fluid channel 1008c connecting those reservoirs 1008a/1008b.

The passage of the sample through the channel 1008c brings the pathogen, if any, present in the sample into contact with the antibody-functionalized graphene flakes 1004. Without being limited to any particular theory, the interaction of the pathogen, e.g., listeria bacteria, with the antibodies to which they can bind can mediate a change in the electrical conductivity (and hence resistance) of the underlying graphene flakes 1004, e.g., via charge transfer or other mechanisms. This change in the electrical conductivity of the graphene flakes 1004 can in turn be measured to detect the presence of the pathogen in the sample under study.

In some embodiments, a four-point measurement technique can be used to measure the resistance of the antibody-functionalized graphene layer in response to exposure thereof to a sample under investigation.

A voltage measuring device, such as the above voltage measuring circuitry 701, can be employed to measure a change in the electrical resistance of the underlying graphene flakes in response to the interaction of a pathogen with the antibodies coupled to the graphene flakes.

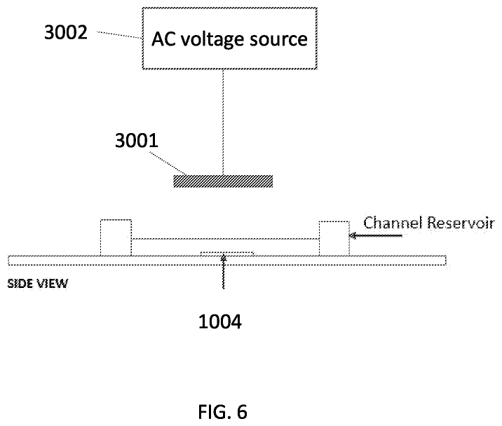

Referring to FIG. 6, in some embodiments, the sensor 1000 according to an embodiment can include a reference electrode 3001 disposed in proximity of the antibody-functionalized graphene flakes 1004 (and/or a graphdiyne layer), e.g., at a distance in a range of about 50 micrometers to about a few millimeters (e.g., 1-2 millimeters) above the antibody-functionalized graphene flakes 1004 (and/or a graphdiyne layer). In some embodiments, rather than being positioned above the graphene flakes layer 1004, the reference electrode can be deposited on a portion of the underlying substrate at a selected distance from the graphene flakes layer, e.g., a distance in a range of about 1 to about 2 mm.

In some embodiments, the distance of the reference electrode 3001 relative to the functionalized graphene flakes 1004 (and/or a graphdiyne layer) can be in a range of about 100 microns to about 1 millimeter, or in a range of about 200 microns to about 0.5 millimeter. Further, as noted above, in some embodiments, rather than being positioned above the graphene flakes 1004 (and/or a graphdiyne layer), the reference electrode 3001 can be positioned in the same plane as the graphene layer.

The reference electrode 3001 can be utilized to generate a time-varying electric field at the interface of the functionalized graphene layer and a liquid sample, e.g., a liquid sample suspected of containing one or more pathogens, that is brought into contact with that layer. For example, in this embodiment, an AC voltage source 3002 can be employed to apply an AC voltage to the reference electrode, which can in turn result in the generation of a time-varying electric field in the space between the reference electrode and the functionalized graphene layer.

The AC reference electrode 3001 can be formed of any suitable electrical conductor. Some examples of suitable conductors include, without limitation, silver, copper, and gold. In some embodiments, the thickness of the reference electrode 3001 can be, for example, in a range of about 100 nm to about 400 micrometers (microns), e.g., in a range of about 1 micron to about 100 microns, though other thicknesses can also be employed.

As discussed above, the application of such a time-varying electric field via the reference electrode 3001 to the interface between the graphene layer and a liquid sample in contact with the graphene layer can advantageously facilitate the detection of one or more electrical properties of the antibody-functionalized graphene flakes 1004, e.g., a change in its resistance in response to its interaction with a pathogen present in the sample that exhibits specific binding to the antibody of the functionalized graphene layer. In particular, it has been discovered that the application of an AC voltage having a frequency in a range of about 1 kHz to about 1 MHz, e.g., in a range of about 10 kHz to about 500 kHz, or in a range of about 20 kHz to about 400 kHz, or in a range of about 30 kHz to about 300 kHz, or in a range of about 40 kHz to about 200 kHz, can be especially advantageous in this regard. By way of example, the amplitude of the AC voltage applied to the reference electrode can be in a range of about 1 millivolt to about 3 volts, e.g., in a range of about 100 millivolts to about 2 volts, or in range of about 200 millivolts to about 1 volt, or in range of about 300 millivolts to about 1 volt, e.g., in a range of about 0.5 volts to 1 volt. Further, in some cases, the voltage applied to the reference electrode can have an AC component and a DC offset, where the DC offset can be in a range of about −40 volts to about +40 volts, e.g., −1 volt to about +1 volt.

Without being limited to any particular theory, in some embodiments, it is expected that the application of such a voltage to the reference electrode can minimize, and preferably eliminate, an effective capacitance associated with a sample, e.g., a liquid sample, with which the functionalized graphene flakes (and/or a graphdiyne layer) are brought into contact as the sample is being tested, thereby facilitating the detection of a change in the resistance of the underlying graphene flakes (and/or a graphdiyne layer) in response to the interaction of the antibodies or aptamers with a respective target analyte (e.g., pathogen). In some cases, the effective capacitance of the sample can be due to ions present in the sample.

The present teachings can be applied to detect a variety of target analytes (e.g., pathogens), such as those discussed above, in a variety of different samples. Some examples of samples that can be interrogated include, without limitation, food samples and bodily fluids, such as blood, urine, saliva, etc.

In some embodiments, a sensor according to the present teachings is capable of detecting target analytes (e.g., pathogens) in a variety of different sample types including, without limitation, urine, mucous and/or blood. In some cases, e.g., when the detection of chlamydia is desired, the sample can be obtained by a swab, e.g., an endocervical swab. Other methods known in the art for obtaining samples can also be utilized.

In another aspect, a sensor according to the present teachings having antibody-functionalized graphene flakes (and/or a graphdiyne layer) can be employed to detect a variety of biomarkers. By way of example, in some embodiments, antibody-functionalized graphene flakes (and/or a graphdiyne layer) can be employed to detect troponin in a biological sample.

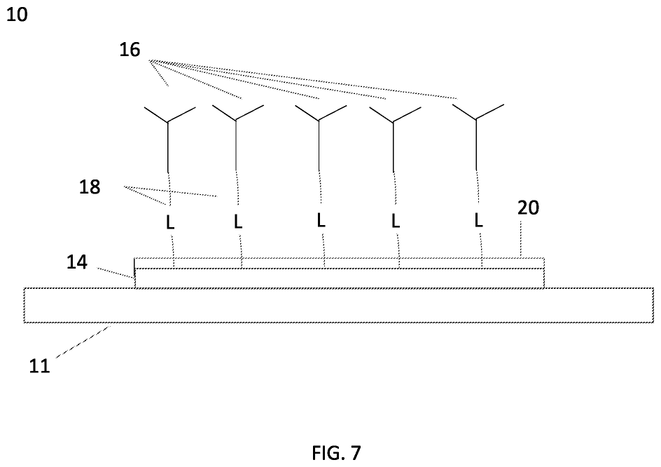

By way of example, FIG. 7 schematically depicts a sensor 10 according to an embodiment of the present teachings for detecting an analyte, e.g., troponin, in a sample, e.g., a patient's blood. The sensor 10 comprises a layer of graphene flakes 14 (and/or a graphdiyne layer), which is disposed on an underlying substrate 11. The underlying substrate 11 can be formed of a variety of different materials, such as, silicon, polymeric materials, such as polyurethane, polyethylene terephthalate, or glass, among others. In some embodiments, the graphene flakes 14 (and/or a graphdiyne layer) are disposed over an underlying silicon oxide (SiO₂) layer, which can in turn be formed as a thin layer in a silicon substrate (e.g., a layer having a thickness in a range of a 200 nm to about 10 microns).

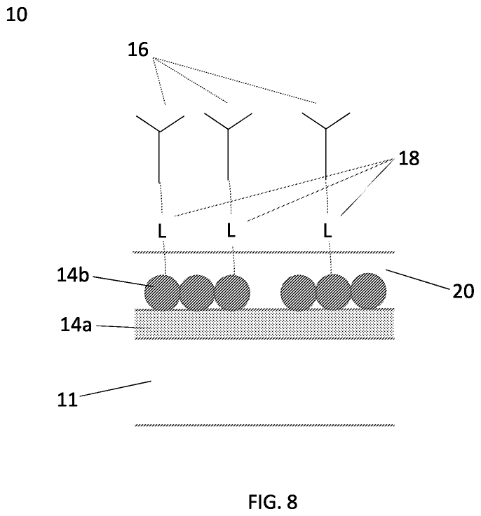

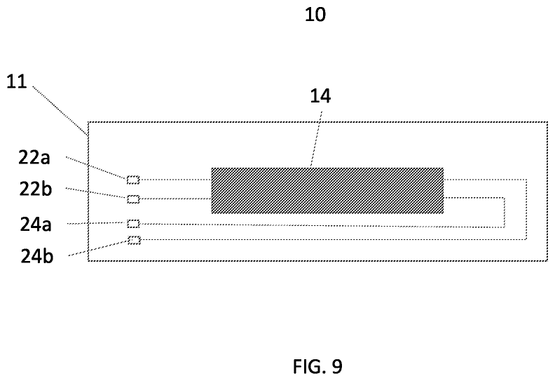

By way of example, FIG. 8 schematically depicts a sensor 10 according to another embodiment of the present teachings. The sensor 10 comprises a multi-layer structure in which a plurality of graphene and/or graphdiyne flakes 14b are deposited on an underlying graphene or graphdiyne layer 14a. Any combination of the flakes 14b and the layer 14a are possible. In other words, a plurality of graphene flakes can be deposited on a graphene layer; a plurality of graphdiyne flakes can be deposited on a graphene layer; a plurality of graphene flakes and a plurality of graphdiyne flakes can be deposited on a graphene layer; a plurality of graphene flakes can be deposited on a graphdiyne layer; a plurality of graphdiyne flakes can be deposited on a graphdiyne layer; a plurality of graphene flakes and a plurality of graphdiyne flakes can be deposited on a graphdiyne layer.

In this embodiment, the graphene flakes 14 (and/or a graphdiyne layer) are functionalized with a plurality of anti-troponin antibodies 16, in other words, in this embodiment, the graphene flakes 14 (and/or a graphdiyne layer) are functionalized with a plurality of antibodies 16 that exhibit, specific binding to troponin, e.g., a particular isoform of troponin. In some embodiments, the anti-troponin antibodies are monoclonal antibodies that exhibit specific binding to a particular isoform of troponin. By way of example, in some embodiments, the graphene flakes 14 (and/or a graphdiyne layer) can be functionalized with cardiac troponin T (cTnT) and/or cardiac troponin I (cTnI).

Similar to the previous embodiments, a variety of linker molecules 18 can be employed for coupling the anti-troponin antibodies 16 to the underlying graphene flakes 14 (and/or a graphdiyne layer). By way of example, in some embodiments, 1-pyrenebutonic acid succinimidyl ester is employed as a linker to facilitate the coupling of the anti-troponin antibodies 16 to the underlying graphene flakes 14 (and/or a graphdiyne layer).

In this embodiment, the plurality of anti-troponin antibodies can cover a fraction of, or the entire, surface of the graphene flakes (and/or a graphdiyne) . In various embodiments, the fraction can be at least about 60%, at least about 70%, at least about 80%, or 100% of the graphene flakes. The remainder of the graphene flakes (i.e., the graphene flakes not functionalized with the anti-troponin antibodies) and/or parts of the substrate that are free of graphene flakes can be passivated via a passivation layer 20. By way of example, the passivation layer can be formed by using Tween 20, BLOTTO, BSA (Bovine Serum Albumin), gelatin and/or amino-PEGS-alcohol (pH 7.4). The passivation layer can inhibit, and preferably prevent, the interaction of a sample of interest introduced onto the antibody-functionalized graphene flakes with those graphene flakes that are not functionalized with the anti-troponin antibodies and/or parts of the substrate that are free of graphene flakes. This can in turn lower the noise in the electrical signals that will be generated as a result of the interaction of the analyte of interest with the antibody molecules.

By way of example, in some embodiments, a plurality of graphene flakes (and/or a graphdiyne layer) deposited on an underlying substrate (e.g., plastic, a semiconductor, such as silicon, or a metal substrate, such as a copper film) can he incubated with the linker molecule (e.g., a 5 mM solution of 1-pyrenebutonic acid succinimidyl ester) for a few hours (e.g., 2 hours) at room temperature.

The linker modified graphene flakes (and/or a graphdiyne layer) can then be incubated with the antibody of interest in a buffer solution (e.g. NaCO₃-NaHCO₃ buffer solution (pH 9)) at a selected temperature and for a selected duration (e.g., 7-10 hours at 4 C), followed by rinsing with deionized (DI) water and phosphate buffered solution (PBS). In order to quench the unreacted succinimidyl ester groups, the modified graphene flakes can be incubated with ethanolaminc (e.g., 0.1 M solution at a pH of 9 for 1 hour).

Similar to the previous embodiments, subsequently, the non-functionalized graphene flakes (and/or a graphdiyine layer) areas can be passivated via a passivation layer, such as the passivation layer 20 schematically depicted in FIG. 7. By way of example, the passivation of the non-functionalized graphene flakes can be achieved, e.g., via incubation with 0.1% Tween 20.

Similar to the previous embodiments and with reference to FIG. 9, the sensor 10 further includes electrically conductive pads 22a, 22b, 24a and 24b, that allow four point measurement of modulation of an electrical property of the functionalized grapheme flakes in response to interaction of anti-troponin antibodies with the troponin coupled to the graphene flakes 14 (and/or a graphdiyne layer). In particular, in this embodiment, the conductive pads 22a/22b are electrically coupled to one end of the functionalized graphene flakes 14 and the conductive pads 24a/24b are electrically coupled to the opposed end of the functionalized graphene flakes 14 to allow measuring a change in an electrical property of the underlying graphene flakes 14 caused by the interaction of troponin in a sample under study with the anti-troponin antibodies that are coupled to the graphene flakes 14 (and/or a graphdiyne layer).

By way of example, in this embodiment, a change in the DC resistance of the underlying graphene flakes 14 (and/or a graphdiyne layer) can be monitored to determine the presence of troponin in a sample under study. In other embodiments, a change in electrical impedance of the graphene flakes 14 (and/or a graphdiyne layer) characterized by a combination of DC resistance and capacitance of the gmphenclantibody system can be monitored to determine whether troponin is present in a sample under study. The electrically conductive pads 22a, 22b, 24a, and 24b can be formed using a variety of metals, such as copper and copper alloys, among others.

An analyzer similar to that described above in connection with FIGS. 2 and 3 can be employed to measure a change in an electrical property of the antibody-functionalized graphene flakes (and/or a graphdiyne layer). The analyzer can further include a module comprising instructions for analyzing the measured electrical signals generated by the functionalized graphene layer to determine whether troponin is present in a sample, and optionally quantify the amount of the troponin in the sample e.g., by comparing the measured electronic signal with a calibration signal. The analyzer can be implemented in hardware, firmware and/or software. By way of example, the analyzer can include a processor in communication, via one or more buses, with one or more memory modules including transient and permanent memory modules. The instructions for analyzing the data received from the sensor can't be stored in at least one of the memory modules and the processor can operate on the data to analyze the signals.

In some embodiments, a sensor according to the present teachings can allow quantifying the amount of troponin, if any, in a sample under study, e.g., a person's blood sample. By way of example, FIG. 10 schematically depicts such a sensor 40 that includes a test sensing element 42 and three calibration sensing elements 44a, 44b, and 44c. Each sensing element includes grapheme flakes functionalized with anti-troponin antibodies and has a structure similar to that discussed above in connection with the sensor 10.

In use, prior to testing a sample with troponin, calibration samples having different concentrations of troponin can be applied to the calibration sensing elements 44a, 44b, and 44c. Each calibration sample includes a different known, amount of troponin, e.g., a known amount of an isoform of troponin, such as cTNT or cTNI. An electrical signal generated by the calibration sensor in response to contact with the calibration sample can be measured and used for quantifying the amount of troponin in a sample under study.

More specifically, each calibration sensing element 44a, 44b, and 44c can be used to obtain an electronic response of the functionalized graphene flakes (and/or a graphdiyne layer) to troponin in one of the calibration samples. The responses of the three calibration sensing elements can be used to generate a calibration curve. While in this embodiment, three calibration sensing elements are employed, in other embodiments, the number of the calibration sensing elements can be more or less than three. A sample under study can be applied to the sample-testing sensing element 42. The calibration curve can be employed to quantify the amount of troponin in the test sample, if any, based on the measured change in the resistance of the underlying graphene layer in response to contact with the test sample.

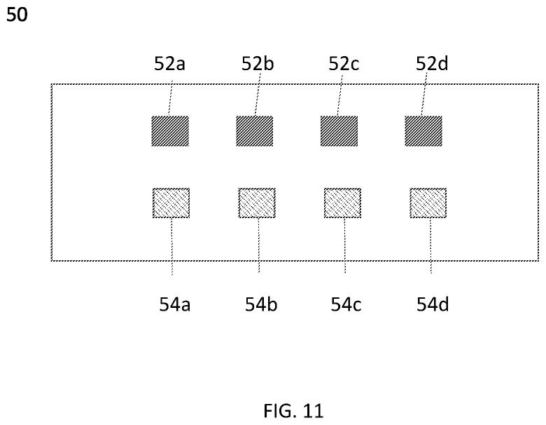

In some embodiments, a sensor according to the present teachings can include an array of sensing elements that allow parallel measurements of different isoforms of troponin. By way of example, FIG. 11 schematically depicts such a sensor 50 haying a plurality of sensing elements 52a, 52b, 52c, and 52d (herein collectively referred to as sensing elements 52) as well as sensing elements 54a, 54b, 54c, and 54d (herein collectively referred to as sensing elements 54). Each of the sensing elements 52 and 54 includes graphene flakes (and/or a graphdiyne layer) that are functionalized with an isoform of troponin and has a structure similar to that discussed above in connection with sensor 10. In this embodiment, each of the sensing elements 52 is functionalized with one isoform of troponin and each of the sensing elements 54 is functionalized with a different isoform of troponin. By way of example, each of the sensing elements 52 can he functionalized with cTNT while each of the sensing elements 54 can be functionalized with cTNI.

In some embodiments, each of the sensing element 52 and 54 can have an associated calibration sensor, which can be employed to calibrate the corresponding sensing element in a manner discussed above.

A sample under study can be concurrently distributed among the sensing elements 52 and 54. The sensing elements 52 can determine whether the sample contains cTNT, and optionally quantify the amount of cTNT in the sample, and the sensing elements 54 can determine whether the sample contains cTNI, and optionally quantify the amount of cTNI in the sample,

An analyzer (not shown), similar to that described above, can be used to measure the change in the resistances of the sensing elements 52 and 54 in response to contact of those sensing elements with a sample under study. In some embodiments, the analyzer can employ a multiplexing circuitry to measure sequentially the resistance of each of the sensing elements.

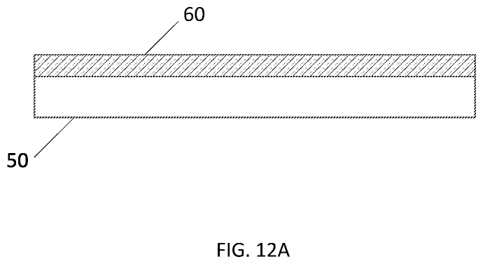

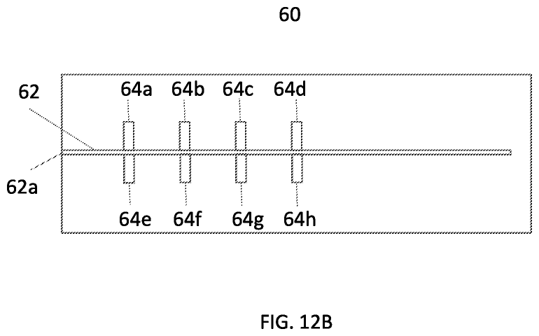

In some embodiments of the sensor 50, a fluidic delivery device can be employed to deliver a sample under study to the sensing elements 52 and 54. By way of example, FIGS. 12A and 12B schematically depict such a fluidic delivery device 60 that is fluidically coupled to the sensing elements 52 and 54. The fluid delivery device 60 includes a central capillary channel 62 having an input port 62a for receiving a sample and a plurality of peripheral capillary channels 64a, 64b, 64c, 64d, 64e, 64f, 64g, and 64h for delivering the sample to the sensing elements 52 and 54.

In some embodiments, one or more of the sensing elements of the sensors can be functionalized with antibodies exhibiting specific binding to C reactive protein (CRP) (herein referred to as anti-CRP antibodies), one or more of the other sensing elements can be functionalized with anti-bodies exhibiting specific binding B-type natriuretic peptide (BNP) (herein referred to as anti-BNP antibodies), and one or more of the sensing elements can be functionalized with anti-troponin, e.g., anti-cTNT, antibodies. A sample under study, e.g., a blood or urine sample, can be introduced onto these sensing elements. One or more electrical signals generated by the sensing elements, e.g., signals generated due to a change in the resistance of the underlying graphene layer, in response to the interaction of the sample with the functionalized graphene layer can he detected and analyzed to identify the presence of the above proteins, i.e., CRP, BNP and troponin, in the sample under study.

As discussed above, in some embodiments, each sensing element can have an associated calibration sensing element that allows calibrating the sensing element for quantifying the concentration of a protein in a sample under study. In this manner, a sensor having a plurality of sensing elements functionalized with a variety of different biomarkers, such as the above biomarkers (CRP, B-type natriuretic peptide and cTNT), can be used to not only identify the presence of that biomarker in a sample but also quantify its concentration.

A panel of a plurality of biomarkers can be used as a diagnostic tool for diagnosing one or more disease conditions. Further, in some cases, a panel of biomarkers can be used as a predictive tool. For example, it has been reported that a combination of cTNT, BNP and CRP can be used as a prognostic tool to predict long-term mortality in hemodialysis (HD) patients.

In another embodiment, one or more of the sensing elements of the sensor 50 can be functionalized with anti-bodies exhibiting specific binding to troponin I, e.g., cardiac troponin I, one or more of the other sensing elements can be functionalized with antibodies exhibiting specific binding to myoglobin, e.g., monoclonal or polyclonal anti-myoglobin antibodies available from Invitrogen, and one or more of the sensing elements can be functionalized with antibodies exhibiting specific binding to creatine kinase MB (CK-MB)

A sample, e.g., anticoagulated whole blood or a plasma specimen, can be brought into contact with the sensing elements, e.g., concurrently or sequentially, to determine the level of each of the above proteins, i.e., cTNI, myoglobin and CK-MB, in the specimen. Similar to the previous embodiment, a plurality of calibration sensors functionalized in a manner similar to the testing sensors can he used to quantify the amount of any of these proteins in the sample. By way of example, such a multi-panel test can be used as an aid in the diagnosis of myocardial infarction.

In some embodiments, one or more of the sensing elements of the above sensor 50 can be functionalized with anti-myeloproxidase (anti-MP) antibodies. Such a sensing element can be used in conjunction with other sensing elements, e.g., a sensing element functionalized with anti-CRP antibodies, as an aid in assessing the risk of adverse cardiac events. In sonic embodiments, a sensor according to the present teachings can detect any of the above analytes, e.g., various isoforms of troponin, at a concentration as low as 5 μg/ml or as low as 1 μg/ml, or as low as 1 picogram/ml.

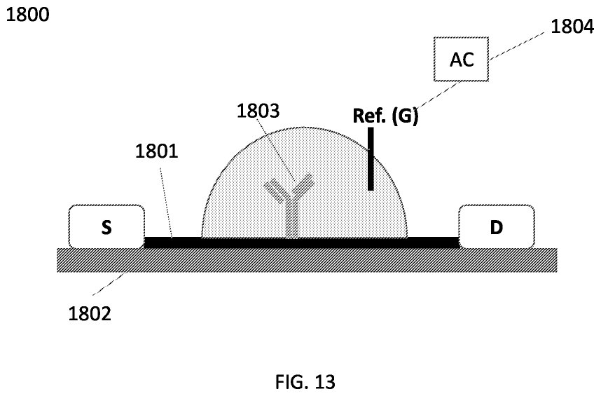

FIG. 13 schematically depicts another embodiment of a sensor 1800 according to the present teachings, which includes a plurality of graphene flakes (and/or a graphdiyne layer) 1801 that are disposed on an underlying substrate 1802, e.g., a semiconductor substrate, and that are functionalized with an antibody of interest 1803. A source electrode (S) and a drain electrode (D) are electrically coupled to the functionalized graphene flakes 1801 (and/or a graphdiyne layer) to allow measuring a change in one or more electrical parameters of the functionalized graphene flakes 1801 (and/or a graphdiyne layer) in response to interaction of the functionalized graphene flakes 1801 (and/or a graphdiyne layer) with a sample. The sensor 1800 further includes a reference electrode (G) that is disposed in proximity of the graphene layer.

In use, in some embodiments, a change in the electrical resistance of the functionalized graphene flakes 1801 (and/or a graphdiyne layer) can be measured in response to the interaction of the functionalized graphene flakes 1801 (and/or a graphdiyne layer) with a sample to identify and optionally, quantify an analyte of interest in the sample. For example, when the sample includes an analyte that specifically binds to the antibody 1803, the interaction of the antibody 1803 with the analyte can modulate the electrical resistance of the graphene flakes 1801. A measurement of such a modulation of the electrical resistance of the grapheme flakes 1801 can be employed to identify that analyte in a sample.

As noted above, it has been discovered that the application of an AC (alternating current) voltage via an AC voltage source 1804 to the graphene flakes 1801 (and/or a graphdiyne layer) can facilitate the detection of one or more electrical properties of the functionalized graphene flakes 1801 (and/or a graphdiyne layer), e.g., a change in its resistance in response to the interaction of the antibody 1803 with an analyte exhibiting specific binding to the antibody 1803. In particular, it has been discovered that the application of an AC voltage having a frequency in a range of about 1 kHz to 1 MHz, e.g., in a range of about 10 kHz to about 500 kHz or in a range of about 20 kHz to about 100 kHz, can be especially advantageous in this regard. By way of example, the amplitude of the AC voltage applied to the reference electrode can be in a range of about 1 millivolt to about 3 volts, e.g., 0.5 volts to 1 volt. Further, in some cases, the voltage applied to the reference electrode can have an AC component and a DC offset, where the DC offset can be in a range of about −40 volts to about +40 volts, e.g., −1 volt to about +1 volt.

Without being limited to any particular theory, in some embodiments, it is expected that the application of a such a voltage to the reference electrode can minimize, and preferably eliminate, an effective capacitance associated with a sample, e.g., a liquid sample, with which the functionalized graphene flakes 1801 (and/or a graphdiyne layer) are brought into contact as the sample is being tested, thereby facilitating the detection of a change in the resistance of the underlying graphene flakes 1801 in response to the interaction of the antibodies 1803 with a respective antigen. In some cases, the effective capacitance of the sample can be due to ions present in the sample.

A sensor according to the present teachings can be employed in a variety of settings. By way of example, a sensor according to the present teachings can be employed in a medical setting. Further, a sensor according to the present teachings can be employed for home use. In such cases, the analyzer can be implemented on a mobile device. In addition or alternatively, the analyzer can be implemented on a remote server that can be in communication with the sensor via a network, e.g., the Internet, to receive sensing data, such as a voltage measured across the antibody-functionalized graphene flakes (and/or a graphdiyne layer). The analyzer can employ the sensing data to determine whether an analyte of interest is present in a sample under study in a manner discussed above.

The graphene flakes (and/or a graphdiyne layer) employed in various embodiments of the present teachings can be fabricated using a variety of different methods. Some such methods rely on bottom-up production of graphene flakes in which small molecular units are combined to form large aromatic hydrocarbons via a variety of chemical reactions. For example, the articles by J. Wu et al. published in Chem. Rev. 107 (2007) 718 and by Zhi et al. published in J. Mater. Chem. 18 (2008) 1472, which are herein incorporated by reference in their entirety, describe such bottom-up methods for fabricating graphene nano-flakes. These articles also describe adding a variety of terminations to these structures, including hydrogen and alkyl groups.

Other methods of generating graphene nano-flakes rely on top-down synthesis techniques. For example, in some such fabrication techniques, a piece of graphene (or graphene-related material such as graphene oxide) can be cut directly into graphene nano-flakes.

The graphdiyne layer and/or the graphdiyne flakes employed in various embodiments of the present teachings can be fabricated using a variety of different methods. Some such methods rely on on-surface synthesis (e.g., on Au, Ag, or Cu surfaces), top-down method, explosion method, and wet chemistry methods. For example, the articles by Gao et al. published in Chem. Soc. Rev. 38 (2019) 908-936 and by Jia et al. published in Acc. Chem. Res. 50 (2017) 343-349, which are herein incorporated by reference in their entirety, describe synthesis of graphdiyne.

In some embodiments, a mixture of graphene nano-flakes and/or graphdiyne flakes and a solvent (e.g., acetone can be spin-coated on a substrate, e.g., silicon or glass substrate, and then subsequently functionalized with a desired antibody.

Those having ordinary skill in the art will appreciate that various changes can be made to the above embodiments without departing from the scope of the invention.

What is claimed is:

1. A sensor for detecting an analyte in a sample, comprising:
a plurality of graphene nano-flakes deposited on an underlying substrate,
a plurality of antibodies coupled to the plurality of graphene nano-flakes to generate a plurality of antibody-functionalized graphene nano-flakes, wherein the antibodies of the plurality of antibodies exhibit specific binding to an analyte, and
a plurality of electrical conductors electrically coupled to the plurality of antibody-functionalized graphene nano-flakes for measuring an electrical property thereof

2. The sensor of claim 1, wherein the analyte comprises a pathogen.

3. The sensor of claim 2, wherein the pathogen comprises any of listeria monocytogene, E. coli, chlamydia, and gonorrhea bacteria.

4. The sensor of claim 1, wherein the analyte comprises an allergen.

5. The sensor of claim 4, wherein the allergen comprises a gluten protein.

6. The sensor of claim 1, wherein the analyte comprises a biomarker.

7. The sensor of claim 6, wherein the biomarker comprises any of troponin, C reactive protein (CRP), and B-type natriuretic peptide (BNP).

8. The sensor of claim 1, wherein the analyte comprises a virus.

9. The sensor of claim 8, wherein the virus comprises a SARS-CoV-2 virus.

10. The sensor of claim 1, further comprising a reference electrode disposed in proximity of the plurality of antibody-functionalized graphene nano-flakes.

11. The sensor of claim 10, further comprising an AC voltage source for applying, an AC voltage to the reference electrode.

12. The sensor of claim 11, wherein the AC voltage has a frequency in a range of about 1 kHz to about 1 MHz.

13. The sensor of claim 12, wherein the AC voltage has an amplitude in a range of about 1.00 millivolts to about 3 volts.

14. The sensor of claim 11, further comprising a DC voltage source for applying a DC offset voltage to the reference electrode.

15. The sensor of claim 14, wherein said DC offset voltage has a range of about −10 V to about 10 V.

16. The sensor of claim 1, wherein the electrical property comprises a DC electrical resistance.

17. A method of detecting an analyte in a sample, comprising:
applying a sample to a plurality of graphene nano-flakes functionalized with an antibody exhibiting specific binding to said analyte,
measuring at least one electrical property of the antibody-functionalized graphene nano-flakes, and
using the at least one electrical property to determine whether an analyte is present in the sample.

18. The method of claim 17, further comprising quantifying the analyte based on a determination that the analyte is present in the sample.

19. The method of claim 17, wherein the sample comprises a biological sample.

20. The method of claim 19, wherein the biological sample comprises any of saliva, urine, and blood.
